(19) United States
(12) Patent Application Publication
(10) Pub. No.: US 2024/0208911 A1
(43) Pub. Date: Jun. 27, 2024

(54) POLYMERIZATION INHIBITORS

(71) Applicant: BASF SE, Ludwigshafen am Rhein (DE)

(72) Inventors: Jan Niclas GORGES, Ludwigshafen am Rhein (DE); Rebecca SURE, Ludwigshafen am Rhein (DE); Friederike FLEISCHHAKER, Ludwigshafen am Rhein (DE); Michael SCHWARZ, Ludwigshafen am Rhein (DE); Christian REIN, Ludwigshafen am Rhein (DE); Friedrich-Georg MARTIN, Ludwigshafen am Rhein (DE)

(21) Appl. No.: 18/287,317

(22) PCT Filed: Apr. 11, 2022
(86) PCT No.: PCT/EP2022/059572

§ 371 Date: Oct. 18, 2023

(30) Foreign Application Priority Data
Apr. 20, 2021 (EP) 21169420.3

(51) Int. Cl. C07D 231/38

(52) U.S. Cl. CPC C07D 231/38

(57) ABSTRACT

The present invention relates to a mixture comprising at least one substituted 4-hydroxypyrazole and at least one polymerizable compound.

The present invention relates to a mixture comprising at least one substituted 4-hydroxypyrazole and at least one polymerizable compound.

Chemical compounds having one or more ethylenically unsaturated groups have a pronounced tendency to free-radical polymerization. Such compounds are therefore also referred to as polymerizable compounds. The tendency of these compounds to free-radical polymerization means that they are used as monomers for the production of polymers. However, the pronounced tendency to free-radical polymerization of these compounds is also a disadvantage in that undesired spontaneous free-radical polymerization can occur during storage and transport and also during chemical and physical processing such as distillation or rectification, particularly under the action of energy such as heat and/or light. Such uncontrolled polymerizations can result in the gradual formation of polymer deposits, for example on heated surfaces, that necessitates removal of the polymer deposits and thus often results in shortened operating times. The uncontrolled polymerizations may even proceed explosively.

During storage and transport and also during chemical and physical processing of ethylenically unsaturated compounds that have a tendency to free-radical polymerization or mixtures that comprise such compounds, it is therefore customary to add compounds that prevent or at least retard undesired spontaneous free-radical polymerization. Such substances are often referred to as polymerization inhibitors.

Polymerization inhibitors may be employed as individual chemical compounds or as mixtures of compounds. Particular requirements are placed on the polymerization inhibitor, depending on its field of use. For a polymerization inhibitor to be suitable as a transport and/or storage stabilizer of ethylenically unsaturated compounds it is important that the efficiency of the polymerization inhibitor, i.e. the extent of inhibition of polymerization, is controllable. Under the conditions of storage and/or transport of the ethylenically unsaturated compounds, the polymerization inhibitor should adequately prevent or retard undesired spontaneous free-radical polymerization, whereas the desired free-radical polymerization of the ethylenically unsaturated compounds should be possible under appropriate polymerization conditions without the need to first have to separate the polymerization inhibitor used during storage and/or transport. If the stabilizer used during storage and/or transport is not separated for the desired polymerization, it is important that it does not adversely affect the desired polymerization, for example unintentionally act as a chain-transfer agent.

In terms of global production volume, acrylic acid is without doubt one of the most important ethylenically unsaturated compounds. During storage and/or transport, acrylic acid is normally stabilized against undesired spontaneous free-radical polymerization with 0.018% to 0.022% by weight of hydroquinone monomethyl ether (MEHQ), based on the amount of acrylic acid. For adequate stabilization of acrylic acid against undesired spontaneous free-radical polymerization using MEHQ, it is necessary that oxygen is dissolved in the acrylic acid in sufficient amounts. Sufficient amounts of oxygen are generally dissolved in the acrylic acid when acrylic acid is stored and/or transported in an atmosphere containing 5% to 21% by volume of oxygen. In the desired polymerization of acrylic acid the content of dissolved oxygen in the acrylic acid is de-creased, thus reducing the efficiency of MEHQ as a polymerization inhibitor enough for acrylic acid to undergo polymerization in the presence of MEHQ.

In addition to use as a polymerization inhibitor for the storage and/or transport of acrylic acid, MEHQ is also used as a polymerization inhibitor for the storage and/or transport of methacrylic acid, acrylic esters and/or methacrylic esters or of mixtures comprising one or more of said compounds.

Its widespread use makes MEHQ one of the most important storage and/or transport stabilizers for polymerizable compounds, especially for acrylic acid, methacrylic acid, acrylic esters and methacrylic esters.

Polymerizable compounds are stored in suitable permanently installed containers, such as storage tanks or other storage vessels (see for example Acrylic Acid, A Summary of Safety and Handling, 4th edition 2013, 7 Bulk Storage Facilities and Accessories). Storage of the polymerizable compounds in the respective containers is preferably for at least one minute, particularly preferably at least 10 minutes, very particularly preferably at least 60 minutes. Although the duration of storage under appropriate conditions is theoretically unlimited, the duration of storage is generally reduced to a minimum for economic reasons. Storage of polymerizable compounds in the respective containers is preferably for not more than 180 days, particularly preferably for not more than 90 days, very particularly preferably for not more than 30 days. Particularly preferred ranges arise from the free combination of the abovementioned lower and upper ranges.

Polymerizable compounds are normally transported in suitable transportable containers such as tanks or other vessels by ship, rail and/or truck (see for example Acrylic Acid, A Summary of Safety and Handling, 4th edition 2013, 9 Safe Transport of Acrylic Acid). The transportable containers may of course also be permanently installed on the corresponding means of transportation, for example tanker ships or rail tank cars. It is of course also possible for containers to be stored for a certain time in a particular place before they are transported to another site. Polymerizable compounds may also be transported in pipelines or hoses, for example to a storage tank after purification of polymerizable compounds, during loading from the storage tank into a transportable container, and/or during loading from one transportable container into another. It is preferable when transport of the polymerizable compounds in the respective containers is for at least 10 seconds, preferably at least one minute, particularly preferably at least 10 minutes, very particularly preferably at least 60 minutes. Although the duration under appropriate conditions is theoretically unlimited, the duration is generally reduced to a minimum for economic and safety reasons. Transport of polymerizable compounds in the respective containers is preferably for not more than 180 days, particularly preferably for not more than 90 days, very particularly preferably for not more than 30 days. Particularly preferred ranges arise from the free combination of the abovementioned lower and upper ranges.

It was an object of the present invention to provide mixtures comprising at least one polymerizable compound, in particular acrylic acid, methacrylic acid, acrylic esters and/or methacrylic esters, and at least one polymerization inhibitor. The polymerization inhibitors shall ensure sufficient stabilization of the polymerizable compounds against undesired free-radical polymerization. However, for the desired free-radical polymerization there shall be no need to remove the polymerization inhibitors from the mixture prior to the polymerization. The polymerization inhibitors therefore shall not act as chain-transfer agents and/or polymerization inhibitors in the desired free-radical polymerization.

The object is achieved by mixtures comprising at least one compound of the general formula (I),

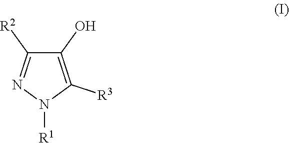

where
R¹ is H or C₁ to C₆ alkyl, preferably H or C₁ to C₄ alkyl,
R² is C₂ to C₅ alkyl, preferably C₂ to C₄ alkyl, particularly preferably C₂ to C₃ alkyl, very particularly preferably C₂ alkyl, and
R³ is C₁ to C₆ alkyl, preferably C₁ to C₄ alkyl, particularly preferably C₁ to C₃ alkyl, very particularly preferably C₁ or C₂ alkyl,
and at least one polymerizable compound.

Examples of suitable compounds of the general formula (I) are 3-ethyl-4-hydroxy-5-methylpyrazole, 3-ethyl-4-hydroxy-1,5-dimethylpyrazole, 1,3-diethyl-4-hydroxy-5-methylpyrazole, 3-ethyl-4-hydroxy-5-methyl-1-n-propylpyrazole, 3-ethyl-4-hydroxy-5-methyl-1-isopropylpyrazole, 1-n-butyl-3-ethyl-4-hydroxy-5-methylpyrazole, 1-isobutyl-3-ethyl-4-hydroxy-5-methylpyrazole, 1-sec-butyl-3-ethyl-4-hydroxy-5-methylpyrazole, 1-tert-butyl-3-ethyl-4-hydroxy-5-methylpyrazole,
4-hydroxy-5-methyl-3-n-propylpyrazole, 4-hydroxy-1,5-dimethyl-3-n-propylpyrazole, 1-ethyl-4-hydroxy-5-methyl-3-n-propylpyrazole, 4-hydroxy-5-methyl-1,3-di-n-propylpyrazole, 4-hydroxy-5-methyl-3-n-propyl-1-isopropylpyrazole, 1-n-butyl-4-hydroxy-5-methyl-3-n-propylpyrazole, 1-iso-butyl-4-hydroxy-5-methyl-3-n-propylpyrazole, 1-sec-butyl-4-hydroxy-5-methyl-3-n-propylpyrazole, 1-tert-butyl-4-hydroxy-5-methyl-3-n-propylpyrazole,
4-hydroxy-5-methyl-3-isopropylpyrazole, 4-hydroxy-1,5-dimethyl-3-isopropylpyrazole, 1-ethyl-4-hydroxy-5-methyl-3-isopropylpyrazole, 4-hydroxy-5-methyl-1-n-propyl-3-isopropylpyrazole, 4-hydroxy-5-methyl-1,3-diisopropylpyrazole, 1-n-butyl-4-hydroxy-5-methyl-3-isopropylpyrazole, 1-isobutyl-4-hydroxy-5-methyl-3-isopropylpyrazole, 1-sec-butyl-4-hydroxy-5-methyl-3-isopropylpyrazole, 1-tert-butyl-4-hydroxy-5-methyl-3-isopropylpyrazole,
3-n-butyl-4-hydroxy-5-methylpyrazole, 3-n-butyl-4-hydroxy-1,5-dimethylpyrazole, 3-n-butyl-1-ethyl-4-hydroxy-5-methylpyrazole, 3-n-butyl-4-hydroxy-5-methyl-1-n-propylpyrazole, 3-n-butyl-4-hydroxy-5-methyl-1-isopropylpyrazole, 1,3-di-n-butyl-4-hydroxy-5-methylpyrazole, 3-n-butyl-1-isobutyl-4-hydroxy-5-methylpyrazole, 3-n-butyl-1-sec-butyl-4-hydroxy-5-methylpyrazole, 3-n-butyl-1-tert-butyl-4-hydroxy-5-methylpyrazole,
3-isobutyl-4-hydroxy-5-methylpyrazole, 3-isobutyl-4-hydroxy-1,5-dimethylpyrazole, 3-isobutyl-1-ethyl-4-hydroxy-5-methylpyrazole, 3-isobutyl-4-hydroxy-5-methyl-1-n-propylpyrazole, 3-isobutyl-4-hydroxy-5-methyl-1-isopropylpyrazole, 1-n-butyl-3-isobutyl-4-hydroxy-5-methylpyrazole, 1,3-diisobutyl-4-hydroxy-5-methylpyrazole, 1-sec-butyl-3-isobutyl-4-hydroxy-5-methylpyrazole, 3-isobutyl-1-tert-butyl-4-hydroxy-5-methylpyrazole,
3-sec-butyl-4-hydroxy-5-methylpyrazole, 3-sec-butyl-4-hydroxy-1,5-dimethylpyrazole, 3-sec-butyl-1-ethyl-4-hydroxy-5-methylpyrazole, 3-sec-butyl-4-hydroxy-5-methyl-1-n-propylpyrazole, 3-sec-butyl-4-hydroxy-5-methyl-1-isopropylpyrazole, 1-n-butyl-3-sec-butyl-4-hydroxy-5-methylpyrazole, 3-sec-butyl-1-isobutyl-4-hydroxy-5-methylpyrazole, 1,3-di-sec-butyl-4-hydroxy-5-methylpyrazole, 3-sec-butyl-1-tert-butyl-4-hydroxy-5-methylpyrazole,
3-tert-butyl-4-hydroxy-5-methylpyrazole, 3-tert-butyl-4-hydroxy-1,5-dimethylpyrazole, 3-tert-bu-tyl-1-ethyl-4-hydroxy-5-methylpyrazole, 3-tert-butyl-4-hydroxy-5-methyl-1-n-propylpyrazole, 3-tert-butyl-4-hydroxy-5-methyl-1-isopropylpyrazole, 1-n-butyl-3-tert-butyl-4-hydroxy-5-methylpyrazole, 1-isobutyl-3-tert-butyl-4-hydroxy-5-methylpyrazole, 1-sec-butyl-3-tert-butyl-4-hydroxy-5-methylpyrazole, 1,3-di-tert-butyl-4-hydroxy-5-methylpyrazole,
3,5-diethyl-4-hydroxypyrazole, 3,5-diethyl-4-hydroxy-1-methylpyrazole, 1,3,5-triethyl-4-hydroxypyrazole, 3,5-diethyl-4-hydroxy-1-n-propylpyrazole, 3,5-diethyl-4-hydroxy-1-isopropylpyrazole, 1-n-butyl-3,5-diethyl-4-hydroxypyrazole, 1-isobutyl-3,5-diethyl-4-hydroxypyrazole, 1-sec-butyl-3,5-diethyl-4-hydroxypyrazole, 1-tert-butyl-3,5-diethyl-4-hydroxypyrazole,
5-ethyl-4-hydroxy-3-n-propylpyrazole, 5-ethyl-4-hydroxy-1-methyl-3-n-propylpyrazole, 1,5-di-ethyl-4-hydroxy-3-n-propylpyrazole, 5-ethyl-4-hydroxy-1,3-di-n-propylpyrazole, 5-ethyl-4-hydroxy-3-n-propyl-1-isopropylpyrazole, 1-n-butyl-5-ethyl-4-hydroxy-3-n-propylpyrazole, 1-isobutyl-5-ethyl-4-hydroxy-3-n-propylpyrazole, 1-sec-butyl-5-ethyl-4-hydroxy-3-n-propylpyrazole, 1-tert-butyl-5-ethyl-4-hydroxy-3-n-propylpyrazole,
5-ethyl-4-hydroxy-3-isopropylpyrazole, 5-ethyl-4-hydroxy-1-methyl-3-isopropylpyrazole, 1,5-di-ethyl-4-hydroxy-3-isopropylpyrazole, 5-ethyl-4-hydroxy-1-n-propyl-3-isopropylpyrazole, 5-ethyl-4-hydroxy-1,3-diisopropylpyrazole, 1-n-butyl-5-ethyl-4-hydroxy-3-isopropylpyrazole, 1-isobutyl-5-ethyl-4-hydroxy-3-isopropylpyrazole, 1-sec-butyl-5-ethyl-4-hydroxy-3-isopropylpyrazole, 1-tert-butyl-5-ethyl-4-hydroxy-3-isopropylpyrazole,
3-n-butyl-5-ethyl-4-hydroxypyrazole, 3-n-butyl-5-ethyl-4-hydroxy-1-methylpyrazole, 3-n-butyl-1,5-diethyl-4-hydroxypyrazole, 3-n-butyl-5-ethyl-4-hydroxy-1-n-propylpyrazole, 3-n-butyl-5-ethyl-4-hydroxy-1-isopropylpyrazole, 1,3-di-n-butyl-5-ethyl-4-hydroxypyrazole, 3-n-butyl-1-isobutyl-5-ethyl-4-hydroxypyrazole, 3-n-butyl-1-sec-butyl-5-ethyl-4-hydroxypyrazole, 3-n-butyl-1-tert-butyl-5-ethyl-4-hydroxypyrazole,
5-ethyl-3-isobutyl-5-ethyl-4-hydroxypyrazole, 3-isobutyl-5-ethyl-4-hydroxy-1-methylpyrazole, 3-isobutyl-1,5-diethyl-4-hydroxypyrazole, 3-isobutyl-5-ethyl-4-hydroxy-1-n-propylpyrazole, 3-isobutyl-5-ethyl-4-hydroxy-1-isopropylpyrazole, 1-n-butyl-3-isobutyl-5-ethyl-4-hydroxypyrazole, 1,3-diisobutyl-5-ethyl-4-hydroxypyrazole, 1-sec-butyl-3-isobutyl-5-ethyl-4-hydroxypyrazole, 3-isobutyl-1-tert-butyl-5-ethyl-4-hydroxypyrazole,
3-sec-butyl-5-ethyl-4-hydroxypyrazole, 3-sec-butyl-5-ethyl-4-hydroxy-1-methylpyrazole, 3-sec-butyl-1,5-diethyl-4-hydroxypyrazole, 3-sec-butyl-5-ethyl-4-hydroxy-1-n-propylpyrazole, 3-sec-butyl-5-ethyl-4-hydroxy-1-isopropylpyrazole, 1-n-butyl-3-sec-butyl-5-ethyl-4-hydroxypyrazole, 3-sec-butyl-1-isobutyl-5-ethyl-4-hydroxypyrazole, 1,3-di-sec-butyl-5-ethyl-4-hydroxypyrazole, 3-sec-butyl-1-tert-butyl-5-ethyl-4-hydroxypyrazole,
3-tert-butyl-5-ethyl-4-hydroxypyrazole, 3-tert-butyl-5-ethyl-4-hydroxy-1-methylpyrazole, 3-tert-butyl-1,5-ethyl-4-hydroxypyrazole, 3-tert-butyl-5-ethyl-4-hydroxy-1-n-propylpyrazole, 3-tert-butyl-5-ethyl-4-hydroxy-1-isopropylpyrazole, 1-n-butyl-3-tert-butyl-5-ethyl-4-hydroxypyrazole, 1-isobutyl-3-tert-butyl-5-ethyl-4-hydroxypyrazole, 1-sec-butyl-3-tert-butyl-5-ethyl-4-hydroxypyrazole, 1,3-di-tert-butyl-5-ethyl-4-hydroxypyrazole,
3-ethyl-4-hydroxy-5-n-propylpyrazole, 3-ethyl-4-hydroxy-1-methyl-5-n-propylpyrazole, 1,3-di-ethyl-4-hydroxy-5-n-propylpyrazole, 3-ethyl-4-hydroxy-1,5-di-n-propylpyrazole, 3-ethyl-4-hydroxy-5-n-propyl-1-isopropylpyrazole, 1-n-butyl-3-ethyl-4-hydroxy-5-n-propylpyrazole, 1-isobutyl-3-ethyl-4-hydroxy-5-n-propylpyrazole, 1-sec-butyl-3-ethyl-4-hydroxy-5-n-propylpyrazole, 1-tert-butyl-3-ethyl-4-hydroxy-5-n-propylpyrazole,
4-hydroxy-3,5-di-n-propylpyrazole, 4-hydroxy-1-methyl-3,5-di-n-propylpyrazole, 1-ethyl-4-hydroxy-3,5-di-n-propylpyrazole, 4-hydroxy-1,3,5-tri-n-propylpyrazole, 4-hydroxy-3,5-di-n-propyl-1-isopropylpyrazole, 1-n-butyl-4-hydroxy-3,5-di-n-propylpyrazole, 1-isobutyl-4-hydroxy-3,5-di-n-propylpyrazole, 1-sec-butyl-4-hydroxy-3,5-di-n-propylpyrazole, 1-tert-butyl-4-hydroxy-3,5-di-n-propylpyrazole,
4-hydroxy-5-n-propyl-3-isopropylpyrazole, 4-hydroxy-1-methyl-5-n-propyl-3-isopropylpyrazole, 1-ethyl-4-hydroxy-5-n-propyl-3-isopropylpyrazole, 4-hydroxy-1,5-di-n-propyl-3-isopropylpyrazole, 4-hydroxy-5-n-propyl-1,3-diisopropylpyrazole, 1-n-butyl-4-hydroxy-5-n-propyl-3-isopropylpyrazole, 1-isobutyl-4-hydroxy-5-n-propyl-3-isopropylpyrazole, 1-sec-butyl-4-hydroxy-5-n-propyl-3-isopropylpyrazole, 1-tert-butyl-4-hydroxy-5-n-propyl-3-isopropylpyrazole,
3-n-butyl-4-hydroxy-5-n-propylpyrazole, 3-n-butyl-4-hydroxy-1-methyl-5-n-propylpyrazole, 3-n-butyl-1-ethyl-4-hydroxy-5-n-propylpyrazole, 3-n-butyl-4-hydroxy-1,5-di-n-propylpyrazole, 3-n-butyl-4-hydroxy-5-n-propyl-1-isopropylpyrazole, 1,3-di-n-butyl-4-hydroxy-5-n-propylpyrazole, 3-n-butyl-1-isobutyl-4-hydroxy-5-n-propylpyrazole, 3-n-butyl-1-sec-butyl-4-hydroxy-5-n-propylpyrazole, 3-n-butyl-1-tert-butyl-4-hydroxy-5-n-propylpyrazole,
3-isobutyl-4-hydroxy-5-n-propylpyrazole, 3-isobutyl-4-hydroxy-1-methyl-5-n-propylpyrazole, 3-isobutyl-1-ethyl-4-hydroxy-5-n-propylpyrazole, 3-isobutyl-4-hydroxy-1,5-di-n-propylpyrazole, 3-isobutyl-4-hydroxy-5-n-propyl-1-isopropylpyrazole, 1-n-butyl-3-isobutyl-4-hydroxy-5-n-propylpyrazole, 1,3-diisobutyl-4-hydroxy-5-n-propylpyrazole, 1-sec-butyl-3-isobutyl-4-hydroxy-5-n-propylpyrazole, 3-isobutyl-1-tert-butyl-4-hydroxy-5-n-propylpyrazole,
3-sec-butyl-4-hydroxy-5-n-propylpyrazole, 3-sec-butyl-4-hydroxy-1-methyl-5-n-propylpyrazole, 3-sec-butyl-1-ethyl-4-hydroxy-5-n-propylpyrazole, 3-sec-butyl-4-hydroxy-1,5-di-n-propylpyrazole, 3-sec-butyl-4-hydroxy-5-n-propyl-1-isopropylpyrazole, 1-n-butyl-3-sec-butyl-4-hydroxy-5-n-propylpyrazole, 3-sec-butyl-1-isobutyl-4-hydroxy-5-n-propylpyrazole, 1,3-di-sec-butyl-4-hydroxy-5-n-propylpyrazole, 3-sec-butyl-1-tert-butyl-4-hydroxy-5-n-propylpyrazole,
3-tert-butyl-4-hydroxy-5-n-propylpyrazole, 3-tert-butyl-4-hydroxy-1-methyl-5-n-propylpyrazole, 3-tert-butyl-1-ethyl-4-hydroxy-5-n-propylpyrazole, 3-tert-butyl-4-hydroxy-1,5-di-n-propylpyrazole, 3-tert-butyl-4-hydroxy-5-n-propyl-1-isopropylpyrazole, 1-n-butyl-3-tert-butyl-4-hydroxy-5-n-propylpyrazole, 1-isobutyl-3-tert-butyl-4-hydroxy-5-n-propylpyrazole, 1-sec-butyl-3-tert-butyl-4-hydroxy-5-n-propylpyrazole, 1,3-di-tert-butyl-4-hydroxy-5-n-propylpyrazole,
3-ethyl-4-hydroxy-5-isopropylpyrazole, 3-ethyl-4-hydroxy-1-methyl-5-isopropylpyrazole, 1,3-di-ethyl-4-hydroxy-5-isopropylpyrazole, 3-ethyl-4-hydroxy-1-n-propyl-5-isopropylpyrazole, 3-ethyl-4-hydroxy-1,5-diisopropylpyrazole, 1-n-butyl-3-ethyl-4-hydroxy-5-isopropylpyrazole, 1-isobutyl-3-ethyl-4-hydroxy-5-isopropylpyrazole, 1-sec-butyl-3-ethyl-4-hydroxy-5-isopropylpyrazole, 1-tert-butyl-3-ethyl-4-hydroxy-5-isopropylpyrazole,
4-hydroxy-3-n-propyl-5-isopropylpyrazole, 4-hydroxy-1-methyl-3-n-propyl-5-isopropylpyrazole, 1-ethyl-4-hydroxy-3-n-propyl-5-isopropylpyrazole, 4-hydroxy-1,3-di-n-propyl-5-isopropylpyrazole, 4-hydroxy-3-n-propyl-1,5-diisopropylpyrazole, 1-n-butyl-4-hydroxy-3-n-propyl-5-isopropylpyrazole, 1-isobutyl-4-hydroxy-3-n-propyl-5-isopropylpyrazole, 1-sec-butyl-4-hydroxy-3-n-propyl-5-isopropylpyrazole, 1-tert-butyl-4-hydroxy-3-n-propyl-5-isopropylpyrazole,
4-hydroxy-3,5-diisopropylpyrazole, 4-hydroxy-1-methyl-3,5-diisopropylpyrazole, 1-ethyl-4-hy-droxy-3,5-diisopropylpyrazole, 4-hydroxy-1-n-propyl-3,5-diisopropylpyrazole, 4-hydroxy-1,3,5-triisopropylpyrazole, 1-n-butyl-4-hydroxy-3,5-diisopropylpyrazole, 1-isobutyl-4-hydroxy-3,5-diisopropylpyrazole, 1-sec-butyl-4-hydroxy-3,5-diisopropylpyrazole, 1-tert-butyl-4-hydroxy-3,5-diisopropylpyrazole,
3-n-butyl-4-hydroxy-5-isopropylpyrazole, 3-n-butyl-4-hydroxy-1-methyl-5-isopropylpyrazole, 3-n-butyl-1-ethyl-4-hydroxy-5-isopropylpyrazole, 3-n-butyl-4-hydroxy-1-n-propyl-5-isopropylpyrazole, 3-n-butyl-4-hydroxy-1,5-diisopropylpyrazole, 1,3-di-n-butyl-4-hydroxy-5-isopropylpyrazole, 3-n-butyl-1-isobutyl-4-hydroxy-5-isopropylpyrazole, 3-n-butyl-1-sec-butyl-4-hydroxy-5-isopropylpyrazole, 3-n-butyl-1-tert-butyl-4-hydroxy-5-isopropylpyrazole,
3-isobutyl-4-hydroxy-5-isopropylpyrazole, 3-isobutyl-4-hydroxy-1-methyl-5-isopropylpyrazole, 3-isobutyl-1-ethyl-4-hydroxy-5-isopropylpyrazole, 3-isobutyl-4-hydroxy-1-n-propyl-5-isopropylpyrazole, 3-isobutyl-4-hydroxy-1,5-diisopropylpyrazole, 1-n-butyl-3-isobutyl-4-hydroxy-5-isopropylpyrazole, 1,3-diisobutyl-4-hydroxy-5-isopropylpyrazole, 1-sec-butyl-3-isobutyl-4-hydroxy-5-isopropylpyrazole, 3-isobutyl-1-tert-butyl-4-hydroxy-5-isopropylpyrazole,
3-sec-butyl-4-hydroxy-5-isopropylpyrazole, 3-sec-butyl-4-hydroxy-1-methyl-5-isopropylpyrazole, 3-sec-butyl-1-ethyl-4-hydroxy-5-isopropylpyrazole, 3-sec-butyl-4-hydroxy-1-n-propyl-5-isopropylpyrazole, 3-sec-butyl-4-hydroxy-1,5-diisopropylpyrazole, 1-n-butyl-3-sec-butyl-4-hydroxy-5-isopropylpyrazole, 3-sec-butyl-1-isobutyl-4-hydroxy-5-isopropylpyrazole, 1,3-di-sec-butyl-4-hydroxy-5-isopropylpyrazole, 3-sec-butyl-1-tert-butyl-4-hydroxy-5-isopropylpyrazole,
3-tert-butyl-4-hydroxy-5-isopropylpyrazole, 3-tert-butyl-4-hydroxy-1-methyl-5-isopropylpyrazole, 3-tert-butyl-1-ethyl-4-hydroxy-5-isopropylpyrazole, 3-tert-butyl-4-hydroxy-1-n-propyl-5-isopropylpyrazole, 3-tert-butyl-4-hydroxy-1,5-diisopropylpyrazole, 1-n-butyl-3-tert-butyl-4-hydroxy-5-isopropylpyrazole, 1-isobutyl-3-tert-butyl-4-hydroxy-5-isopropylpyrazole, 1-sec-butyl-3-tert-bu-tyl-4-hydroxy-5-isopropylpyrazole, 1,3-di-tert-butyl-4-hydroxy-5-isopropylpyrazole,
5-n-butyl-3-ethyl-4-hydroxypyrazole, 5-n-butyl-3-ethyl-4-hydroxy-1-methylpyrazole, 5-n-butyl-1,3-diethyl-4-hydroxypyrazole, 5-n-butyl-3-ethyl-4-hydroxy-1-n-propylpyrazole, 5-n-butyl-3-ethyl-4-hydroxy-1-isopropylpyrazole, 1,5-di-n-butyl-3-ethyl-4-hydroxypyrazole, 5-n-butyl-1-isobutyl-3-ethyl-4-hydroxypyrazole, 5-n-butyl-1-sec-butyl-3-ethyl-4-hydroxypyrazole, 5-n-butyl-1-tert-butyl-3-ethyl-4-hydroxypyrazole,
5-n-butyl-4-hydroxy-3-n-propylpyrazole, 5-n-butyl-4-hydroxy-1-methyl-3-n-propylpyrazole, 5-n-butyl-1-ethyl-4-hydroxy-3-n-propylpyrazole, 5-n-butyl-4-hydroxy-1,3-di-n-propylpyrazole, 5-n-bu-tyl-4-hydroxy-3-n-propyl-1-isopropylpyrazole, 1,5-di-n-butyl-4-hydroxy-3-n-propylpyrazole, 5-n-butyl-1-isobutyl-4-hydroxy-3-n-propylpyrazole, 5-n-butyl-1-sec-butyl-4-hydroxy-3-n-propylpyrazole, 5-n-butyl-1-tert-butyl-4-hydroxy-3-n-propylpyrazole,
5-n-butyl-4-hydroxy-3-isopropylpyrazole, 5-n-butyl-4-hydroxy-1-methyl-3-isopropylpyrazole, 5-n-butyl-1-ethyl-4-hydroxy-3-isopropylpyrazole, 5-n-butyl-4-hydroxy-1-n-propyl-3-isopropylpyrazole, 5-n-butyl-4-hydroxy-1,3-diisopropylpyrazole, 1,5-di-n-butyl-4-hydroxy-3-isopropylpyrazole, 5-n-butyl-1-isobutyl-4-hydroxy-3-isopropylpyrazole, 5-n-butyl-1-sec-butyl-4-hydroxy-3-isopropylpyrazole, 5-n-butyl-1-tert-butyl-4-hydroxy-3-isopropylpyrazole,
3,5 di-n-butyl-4-hydroxypyrazole, 3,5-di-n-butyl-4-hydroxy-1-methylpyrazole, 3,5-di-n-butyl-1-ethyl-4-hydroxypyrazole, 3,5-di-n-butyl-4-hydroxy-1-n-propylpyrazole, 3,5-di-n-butyl-4-hydroxy-1-isopropylpyrazole, 1,3,5-tri-n-butyl-4-hydroxypyrazole, 3,5-di-n-butyl-1-isobutyl-4-hydroxypyrazole, 3,5-di-n-butyl-1-sec-butyl-4-hydroxypyrazole, 3,5-di-n-butyl-1-tert-butyl-4-hydroxypyrazole,
5-n-butyl-3-isobutyl-4-hydroxypyrazole, 5-n-butyl-3-isobutyl-4-hydroxy-1-methylpyrazole, 5-n-butyl-3-isobutyl-1-ethyl-4-hydroxypyrazole, 5-n-butyl-3-isobutyl-4-hydroxy-1-n-propylpyrazole, 5-n-butyl-3-isobutyl-4-hydroxy-1-isopropylpyrazole, 1,5-di-n-butyl-3-isobutyl-4-hydroxypyrazole, 5-n-butyl-1,3-diisobutyl-4-hydroxypyrazole, 5-n-butyl-1-sec-butyl-3-isobutyl-4-hydroxypyrazole, 5-n-butyl-3-isobutyl-1-tert-butyl-4-hydroxypyrazole,
5-n-butyl-3-sec-butyl-4-hydroxypyrazole, 5-n-butyl-3-sec-butyl-4-hydroxy-1-methylpyrazole, 5-n-butyl-3-sec-butyl-1-ethyl-4-hydroxypyrazole, 5-n-butyl-3-sec-butyl-4-hydroxy-1-n-propylpyrazole, 5-n-butyl-3-sec-butyl-4-hydroxy-1-isopropylpyrazole, 1,5-di-n-butyl-3-sec-butyl-4-hydroxypyrazole, 5-n-butyl-3-sec-butyl-1-isobutyl-4-hydroxypyrazole, 5-n-butyl-1,3-di-sec-butyl-4-hydroxy-5-methylpyrazole, 5-n-butyl-3-sec-butyl-1-tert-butyl-4-hydroxypyrazole,
5-n-butyl-3-tert-butyl-4-hydroxypyrazole, 5-n-butyl-3-tert-butyl-4-hydroxy-1-methylpyrazole, 5-n-butyl-3-tert-butyl-1-ethyl-4-hydroxypyrazole, 5-n-butyl-3-tert-butyl-4-hydroxy-1-n-propylpyrazole, 5-n-butyl-3-tert-butyl-4-hydroxy-1-isopropylpyrazole, 1,5-di-n-butyl-3-tert-butyl-4-hydroxypyrazole, 5-n-butyl-1-isobutyl-3-tert-butyl-4-hydroxypyrazole, 5-n-butyl-1-sec-butyl-3-tert-butyl-4-hydroxypyrazole, 5-n-butyl-1,3-di-tert-butyl-4-hydroxypyrazole,
5-sec-butyl-3-ethyl-4-hydroxypyrazole, 5-sec-butyl-3-ethyl-4-hydroxy-1-methylpyrazole, 5-sec-butyl-1,3-diethyl-4-hydroxypyrazole, 5-sec-butyl-3-ethyl-4-hydroxy-1-n-propylpyrazole, 5-sec-butyl-3-ethyl-4-hydroxy-1-isopropylpyrazole, 1-n-butyl-5-sec-butyl-3-ethyl-4-hydroxypyrazole, 5-sec-butyl-1-isobutyl-3-ethyl-4-hydroxypyrazole, 1,5-di-sec-butyl-3-ethyl-4-hydroxypyrazole, 5-sec-butyl-1-tert-butyl-3-ethyl-4-hydroxypyrazole,
5-sec-butyl-4-hydroxy-3-n-propylpyrazole, 5-sec-butyl-4-hydroxy-1-methyl-3-n-propylpyrazole, 5-sec-butyl-1-ethyl-4-hydroxy-3-n-propylpyrazole, 5-sec-butyl-4-hydroxy-1,3-di-n-propylpyrazole, 5-sec-butyl-4-hydroxy-3-n-propyl-1-isopropylpyrazole, 1-n-butyl-5-sec-butyl-4-hydroxy-3-n-propylpyrazole, 5-sec-butyl-1-isobutyl-4-hydroxy-3-n-propylpyrazole, 1,5-di-sec-butyl-4-hydroxy-3-n-propylpyrazole, 5-sec-butyl-1-tert-butyl-4-hydroxy-3-n-propylpyrazole,
5-sec-butyl-4-hydroxy-3-isopropylpyrazole, 5-sec-butyl-4-hydroxy-1-methyl-3-isopropylpyrazole, 5-sec-butyl-1-ethyl-4-hydroxy-3-isopropylpyrazole, 5-sec-butyl-4-hydroxy-1-n-propyl-3-isopropylpyrazole, 5-sec-butyl-4-hydroxy-1,3-diisopropylpyrazole, 1-n-butyl-5-sec-butyl-4-hydroxy-3-isopropylpyrazole, 5-sec-butyl-1-isobutyl-4-hydroxy-3-isopropylpyrazole, 1,5-di-sec-butyl-4-hydroxy-3-isopropylpyrazole, 5-sec-butyl-1-tert-butyl-4-hydroxy-3-isopropylpyrazole, 3-n-butyl-5-sec-butyl-4-hydroxypyrazole, 3-n-butyl-5-sec-butyl-4-hydroxy-1-methylpyrazole, 3-n-butyl-5-sec-butyl-1-ethyl-4-hydroxypyrazole, 3-n-butyl-5-sec-butyl-4-hydroxy-1-n-propylpyrazole, 3-n-butyl-5-sec-butyl-4-hydroxy-1-isopropylpyrazole, 1,3-di-n-butyl-5-sec-butyl-4-hydroxypyrazole, 3-n-butyl-5-sec-butyl-1-isobutyl-4-hydroxypyrazole, 3-n-butyl-1,5-di-sec-butyl-4-hydroxypyrazole, 3-n-butyl-5-sec-butyl-1-tert-butyl-4-hydroxypyrazole,
5-sec-butyl-3-isobutyl-4-hydroxypyrazole, 5-sec-butyl-3-isobutyl-4-hydroxy-1-methylpyrazole, 5-sec-butyl-3-isobutyl-1-ethyl-4-hydroxypyrazole, 5-sec-butyl-3-isobutyl-4-hydroxy-1-n-propylpyrazole, 5-sec-butyl-3-isobutyl-4-hydroxy-1-isopropylpyrazole, 1-n-butyl-5-sec-butyl-3-isobutyl-4-hydroxypyrazole, 5-sec-butyl-1,3-diisobutyl-4-hydroxypyrazole, 1,5-di-sec-butyl-3-isobutyl-4-hydroxypyrazole, 5-sec-butyl-3-isobutyl-1-tert-butyl-4-hydroxypyrazole,
3,5-di-sec-butyl-4-hydroxypyrazole, 3,5-di-sec-butyl-4-hydroxy-1-methylpyrazole, 3,5-di-sec-bu-tyl-1-ethyl-4-hydroxypyrazole, 3,5-di-sec-butyl-4-hydroxy-1-n-propylpyrazole, 3,5-di-sec-butyl-4-hydroxy-1-isopropylpyrazole, 1-n-butyl-3,5-di-sec-butyl-4-hydroxypyrazole, 3,5-di-sec-butyl-1-isobutyl-4-hydroxypyrazole, 1,3,5-tri-sec-butyl-4-hydroxy-5-methylpyrazole, 3,5-di-sec-butyl-1-tert-butyl-4-hydroxypyrazole,
5-sec-butyl-3-tert-butyl-4-hydroxypyrazole, 5-sec-butyl-3-tert-butyl-4-hydroxy-1-methylpyrazole, 5-sec-butyl-3-tert-butyl-1-ethyl-4-hydroxypyrazole, 5-sec-butyl-3-tert-butyl-4-hydroxy-1-n-propylpyrazole, 5-sec-butyl-3-tert-butyl-4-hydroxy-1-isopropylpyrazole, 1-n-butyl-5-sec-butyl-3-tert-butyl-4-hydroxypyrazole, 5-sec-butyl-1-isobutyl-3-tert-butyl-4-hydroxypyrazole, 1,5-di-sec-butyl-3-tert-butyl-4-hydroxypyrazole, 5-sec-butyl-1,3-di-tert-butyl-4-hydroxypyrazole,
5-isobutyl-3-ethyl-4-hydroxypyrazole, 5-isobutyl-3-ethyl-4-hydroxy-1-methylpyrazole, 5-isobutyl-1,3-diethyl-4-hydroxypyrazole, 5-isobutyl-3-ethyl-4-hydroxy-1-n-propylpyrazole, 5-isobutyl-3-ethyl-4-hydroxy-1-isopropylpyrazole, 1-n-butyl-5-isobutyl-3-ethyl-4-hydroxypyrazole, 1,5-diisobu-tyl-3-ethyl-4-hydroxypyrazole, 1-sec-butyl-5-isobutyl-3-ethyl-4-hydroxypyrazole, 5-isobutyl-1-tert-butyl-3-ethyl-4-hydroxypyrazole,
5-isobutyl-4-hydroxy-3-n-propylpyrazole, 5-isobutyl-4-hydroxy-1-methyl-3-n-propylpyrazole, 5-isobutyl-1-ethyl-4-hydroxy-3-n-propylpyrazole, 5-isobutyl-4-hydroxy-1,3-di-n-propylpyrazole, 5-isdobutyl-4-hydroxy-3-n-propyl-1-isopropylpyrazole, 1-n-butyl-5-isobutyl-4-hydroxy-3-n-propylpyrazole, 1,5-diisobutyl-4-hydroxy-3-n-propylpyrazole, 1-sec-butyl-5-isobutyl-4-hydroxy-3-n-propylpyrazole, 5-isobutyl-1-tert-butyl-4-hydroxy-3-n-propylpyrazole,
5-isobutyl-4-hydroxy-3-isopropylpyrazole, 5-isobutyl-4-hydroxy-1-methyl-3-isopropylpyrazole, 5-isobutyl-1-ethyl-4-hydroxy-3-isopropylpyrazole, 5-isobutyl-4-hydroxy-1-n-propyl-3-isopropylpyrazole, 5-isobutyl-4-hydroxy-1,3-diisopropylpyrazole, 1-n-butyl-5-isobutyl-4-hydroxy-3-isopropylpyrazole, 1,5-diisobutyl-4-hydroxy-3-isopropylpyrazole, 1-sec-butyl-5-isobutyl-4-hydroxy-3-isopropylpyrazole, 5-isobutyl-1-tert-butyl-4-hydroxy-3-isopropylpyrazole,
3-n-butyl-5-isobutyl-4-hydroxypyrazole, 3-n-butyl-5-isobutyl-4-hydroxy-1-methylpyrazole, 3-n-butyl-5-isobutyl-1-ethyl-4-hydroxypyrazole, 3-n-butyl-5-isobutyl-4-hydroxy-1-n-propylpyrazole, 3-n-butyl-5-isobutyl-4-hydroxy-1-isopropylpyrazole, 1,3-di-n-butyl-5-isobutyl-4-hydroxypyrazole, 3-n-butyl-1,5-diisobutyl-4-hydroxypyrazole, 3-n-butyl-1-sec-butyl-5-isobutyl-4-hydroxypyrazole, 3-n-butyl-5-isobutyl-1-tert-butyl-4-hydroxypyrazole,
3,5-diisobutyl-4-hydroxypyrazole, 3,5-diisobutyl-4-hydroxy-1-methylpyrazole, 3,5-diisobutyl-1-ethyl-4-hydroxypyrazole, 3,5-diisobutyl-4-hydroxy-1-n-propylpyrazole, 3,5-diisobutyl-4-hydroxy-1-isopropylpyrazole, 1-n-butyl-3,5-diisobutyl-4-hydroxypyrazole, 1,3,5-triisobutyl-4-hydroxypyrazole, 1-sec-butyl-3,5-disobutyl-4-hydroxypyrazole, 3,5-diisobutyl-1-tert-butyl-4-hydroxypyrazole,
3-sec-butyl-5-isobutyl-4-hydroxypyrazole, 3-sec-butyl-5-isobutyl-4-hydroxy-1-methylpyrazole, 3-sec-butyl-5-isobutyl-1-ethyl-4-hydroxypyrazole, 3-sec-butyl-5-isobutyl-4-hydroxy-1-n-propylpyrazole, 3-sec-butyl-5-isobutyl-4-hydroxy-1-isopropylpyrazole, 1-n-butyl-3-sec-butyl-5-isobutyl-4-hydroxypyrazole, 3-sec-butyl-1,5-diisobutyl-4-hydroxypyrazole, 1,3-di-sec-butyl-5-isobutyl-4-hy-droxy-5-methylpyrazole, 3-sec-butyl-5-isobutyl-1-tert-butyl-4-hydroxypyrazole,
5-isobutyl-3-tert-butyl-4-hydroxypyrazole, 5-isobutyl-3-tert-butyl-4-hydroxy-1-methylpyrazole, 5-isobutyl-3-tert-butyl-1-ethyl-4-hydroxypyrazole, 5-isobutyl-3-tert-butyl-4-hydroxy-1-n-propylpyrazole, 5-isobutyl-3-tert-butyl-4-hydroxy-1-isopropylpyrazole, 1-n-butyl-5-isobutyl-3-tert-butyl-4-hydroxypyrazole, 1,5-diisobutyl-3-tert-butyl-4-hydroxypyrazole, 1-sec-butyl-5-isobutyl-3-tert-bu-tyl-4-hydroxypyrazole, 5-isobutyl-1,3-di-tert-butyl-4-hydroxypyrazole,
5-tert-butyl-3-ethyl-4-hydroxypyrazole, 5-tert-butyl-3-ethyl-4-hydroxy-1-methylpyrazole, 5-tert-butyl-1,3-diethyl-4-hydroxypyrazole, 5-tert-butyl-3-ethyl-4-hydroxy-1-n-propylpyrazole, 5-tert-bu-tyl-3-ethyl-4-hydroxy-1-isopropylpyrazole, 1-n-butyl-5-tert-butyl-3-ethyl-4-hydroxypyrazole, 1-isobutyl-5-tert-butyl-3-ethyl-4-hydroxypyrazole, 1-sec-butyl-5-tert-butyl-3-ethyl-4-hydroxypyrazole, 1,5-di-tert-butyl-3-ethyl-4-hydroxypyrazole,
5-tert-butyl-4-hydroxy-3-n-propylpyrazole, 5-tert-butyl-4-hydroxy-1-methyl-3-n-propylpyrazole, 5-tert-butyl-1-ethyl-4-hydroxy-3-n-propylpyrazole, 5-tert-butyl-4-hydroxy-1,3-di-n-propylpyrazole, 5-tert-butyl-4-hydroxy-3-n-propyl-1-isopropylpyrazole, 1-n-butyl-5-tert-butyl-4-hydroxy-3-n-propylpyrazole, 1-isobutyl-5-tert-butyl-4-hydroxy-3-n-propylpyrazole, 1-sec-butyl-5-tert-butyl-4-hydroxy-3-n-propylpyrazole, 1,5-di-tert-butyl-4-hydroxy-3-n-propylpyrazole,
5-tert-butyl-4-hydroxy-3-isopropylpyrazole, 5-tert-butyl-4-hydroxy-1-methyl-3-isopropylpyrazole, 5-tert-butyl-1-ethyl-4-hydroxy-3-isopropylpyrazole, 5-tert-butyl-4-hydroxy-1-n-propyl-3-isopropylpyrazole, 5-tert-butyl-4-hydroxy-1,3-diisopropylpyrazole, 1-n-butyl-5-tert-butyl-4-hydroxy-3-isopropylpyrazole, 1-isobutyl-5-tert-butyl-4-hydroxy-3-isopropylpyrazole, 1-sec-butyl-5-tert-butyl 4-hydroxy-3-isopropylpyrazole, 1,5-di-tert-butyl-4-hydroxy-3-isopropylpyrazole,
3-n-butyl-5-tert-butyl-4-hydroxypyrazole, 3-n-butyl-5-tert-butyl-4-hydroxy-1-methylpyrazole, 3-n-butyl-5-tert-butyl-1-ethyl-4-hydroxypyrazole, 3-n-butyl-5-tert-butyl-4-hydroxy-1-n-propylpyrazole, 3-n-butyl-5-tert-butyl-4-hydroxy-1-isopropylpyrazole, 1,3-di-n-butyl-5-tert-butyl-4-hydroxypyrazole, 3-n-butyl-1-isobutyl-5-tert-butyl-4-hydroxypyrazole, 3-n-butyl-1-sec-butyl-5-tert-butyl-4-hydroxypyrazole, 3-n-butyl-1,5-di-tert-butyl-4-hydroxypyrazole,
3-isobutyl-5-tert-butyl-4-hydroxypyrazole, 3-isobutyl-5-tert-butyl-4-hydroxy-1-methylpyrazole, 3-isobutyl-5-tert-butyl-1-ethyl-4-hydroxypyrazole, 3-isobutyl-5-tert-butyl-4-hydroxy-1-n-propylpyrazole, 3-isobutyl-5-tert-butyl-4-hydroxy-1-isopropylpyrazole, 1-n-butyl-3-isobutyl-5-tert-butyl-4-hydroxypyrazole, 1,3-diisobutyl-5-tert-butyl-4-hydroxypyrazole, 1-sec-butyl-3-isobutyl-5-tert-bu-tyl-4-hydroxypyrazole, 3-isobutyl-1,5-di-tert-butyl-4-hydroxypyrazole,
3-sec-butyl-5-tert-butyl-4-hydroxypyrazole, 3-sec-butyl-5-tert-butyl-4-hydroxy-1-methylpyrazole, 3-sec-butyl-5-tert-butyl-1-ethyl-4-hydroxypyrazole, 3-sec-butyl-5-tert-butyl-4-hydroxy-1-n-propylpyrazole, 3-sec-butyl-5-tert-butyl-4-hydroxy-1-isopropylpyrazole, 1-n-butyl-3-sec-butyl-5-tert-butyl-4-hydroxypyrazole, 3-sec-butyl-1-isobutyl-5-tert-butyl-4-hydroxypyrazole, 1,3-di-sec-butyl-5-tert-butyl-4-hydroxy-5-methylpyrazole, 3-sec-butyl-1,5-di-tert-butyl-4-hydroxypyrazole,
3,5-di-tert-butyl-4-hydroxypyrazole, 3,5-di-tert-butyl-4-hydroxy-1-methylpyrazole, 3,5-di-tert-bu-tyl-1-ethyl-4-hydroxypyrazole, 3,5-di-tert-butyl-4-hydroxy-1-n-propylpyrazole, 3,5-di-tert-butyl-4-hydroxy-1-isopropylpyrazole, 1-n-butyl-3,5-di-tert-butyl-4-hydroxypyrazole, 1-isobutyl-3,5-di-tert-butyl-4-hydroxypyrazole, 1-sec-butyl-3,5-di-tert-butyl-4-hydroxypyrazole, 1,3,5-tri-tert-butyl-4-hydroxypyrazole.

In the mixture the total amount of compounds of the general formula (I) is preferably from 0.0001% to 0.1000% by weight, more preferably from 0.0002% to 0.0750% by weight, particularly preferably from 0.0005% to 0.0500% by weight, very particularly preferably from 0.0010% to 0.0250% by weight, in each case based on the total amount of polymerizable compounds.

In the mixture the total amount of polymerizable compounds is preferably at least 5% by weight, more preferably at least 50% by weight, particularly preferably at least 80% by weight, very particularly preferably at least 99% by weight, in each case based on the total weight of the mixture.

Polymerizable compounds are preferably mono-, di- or triethylenically unsaturated C₃ to C₈ carboxylic acids, mono-, di- or triethylenically unsaturated C₃ to C₈ aldehydes, mono-, di- or triethylenically unsaturated C₃ to C₈ carboxylic esters having 1 to 20 carbon atoms in the ester groups, mono-, di- or triethylenically unsaturated C₃ to C₈ carboxamides, mono-, di- or triethylenically unsaturated C₃ to C₈ nitriles, mono-, di- or triethylenically unsaturated C₃ to C₈ carboxylic anhydrides, vinyl esters of saturated C₂ to C₂₀ carboxylic acids, vinyl ethers of saturated C₁ to C₁₀ alcohols, vinyl aromatics, vinyl heteroaromatics, vinyl lactams having 3 to 10 carbon atoms in the ring, open-chain N-vinylamide compounds and N-vinylamine compounds, vinyl hal-ides, aliphatic, optionally halogenated, hydrocarbons having 2 to 8 carbon atoms and 1 or 2 eth-ylenic double bonds, vinylidenes or any desired mixtures of two or more of the abovementioned compounds.

Particular preference is given to mono-, di- or triethylenically unsaturated C₃ to C₈ carboxylic acids, for example acrylic acid, methacrylic acid, dimethacrylic acid, ethacrylic acid, citraconic acid, methylenemalonic acid, crotonic acid, fumaric acid, mesaconic acid, itaconic acid, and maleic acid, mono-, di- or triethylenically unsaturated C₃ to C₈ carboxylic esters having 1 to 20 carbon atoms in the ester groups, for example acrylic esters with C₁ to C₂₀ alkyl, methacrylic esters with C₁ to C₂₀ alkyl, dimethacrylic esters with C₁ to C₂₀ alkyl, ethacrylic esters with C₁ to C₂₀ alkyl, citraconic esters with C₁ to C₂₀ alkyl, methylenemalonic esters with C₁ to C₂₀ alkyl, crotonic esters with C₁ to C₂₀ alkyl, fumaric esters with C₁ to C₂₀ alkyl, mesaconic esters with C₁ to C₂₀ alkyl, itaconic esters with C₁ to C₂₀ alkyl and maleic esters with C₁ to C₂₀ alkyl, mono-, di- or triethylenically unsaturated C₃ to C₈ carboxamides, for example acrylamide, methacrylamide, di-methacrylamide, ethacrylamide, citraconamide, methylenemalonamide, crotonamide, fumara-mide, mesaconamide, itaconamide, and maleamide, mono-, di- or triethylenically unsaturated C₃ to C₈ nitriles, for example acrylonitrile and methacrylonitrile, and mono-, di- or triethylenically unsaturated C₃ to C₈ carboxylic anhydrides, for example acrylic anhydride, methacrylic anhydride, itaconic anhydride, and maleic anhydride.

Particular preference is given to acrylic acid, methacrylic acid, acrylic esters with C₁ to C₈ alkyl, such as methyl acrylate, ethyl acrylate, n-butyl acrylate, and 2-ethylhexyl acrylate, and methacrylic esters with C₁ to C₈ alkyl, such as methyl methacrylate.

Further suitable polymerizable compounds are dipropylene glycol diacrylate, tripropylene glycol diacrylate, polyethylene glycol diacrylate, glycerol triacrylate, ethoxylated glycerol triacrylate, trimethylolpropane triacrylate, ethoxylated trimethylolpropane triacrylate, butanediol monoacry-late, dicyclopentadienyl acrylate, 2-dimethylaminoethyl acrylate, 2-hydroxyethyl acrylate, and 2-hydroxypropyl acrylate.

With acrylic acid as the polymerizable compound, the total amount of compounds of the general formula (I) in the mixture is preferably from 0.0050% to 0.1000% by weight, more preferably from 0.0100% to 0.0750% by weight, particularly preferably from 0.0120% to 0.0500% by weight, very particularly preferably from 0.0150% to 0.0250% by weight, in each case based on acrylic acid.

With methyl acrylate, methyl methacrylate, ethyl acrylate, n-butyl acrylate and/or 2-ethylhexyl acrylate as the polymerizable compound, the total amount of compounds of the general formula (I) in the mixture is preferably from 0.0001% to 0.0100% by weight, more preferably from 0.0002% to 0.0075% by weight, particularly preferably from 0.0005% to 0.0050% by weight, very particularly preferably from 0.0010% to 0.0020% by weight, in each case based on the total amount of methyl acrylate, methyl methacrylate, ethyl acrylate, n-butyl acrylate and/or 2-ethylhexyl acrylate.

The present invention further provides for the use of at least one compound of the general formula (I) as defined hereinabove, for inhibition of polymerization of at least one polymerizable compound.

The present invention further provides a process for the storage and/or transport of a mixture comprising at least one compound of the general formula (I) as defined hereinabove and at least one polymerizable compound.

The mixture is preferably stored and/or transported in an oxygen-containing atmosphere.

It is preferable when the mixture is stored in a vessel in an oxygen-containing atmosphere having an oxygen content of 5% to 10% by volume and the mixture in the vessel is regularly recirculated, for example by pumped recirculation of the entirety of the tank contents at least once a week. The relatively low oxygen content prevents the formation of ignitable gas mixtures in the vessel. The recirculation replaces consumed dissolved oxygen in the liquid polymerizable compound.

Mixtures of the invention having acrylic acid as the polymerizable compound are particularly suitable for the production of water-swellable polyacrylic acids (superabsorbers) and water-solu-ble polyacrylic acids. The production of superabsorbents is described for example in Ullmann's Encyclopedia of Industrial Chemistry, 6th edition, volume 35, pages 73 to 93.

Mixtures of the invention having methyl acrylate, ethyl acrylate, n-butyl acrylate or 2-ethylhexyl acrylate as the polymerizable compound are particularly suitable for the production of polymer dispersions.

### EXAMPLES

Preparation of 3-ethyl-4-hydroxy-1,5-dimethylpyrazole

Variant A: A solution of 24 g (0.511 mol) of methylhydrazine, 44 g (0.734 mol) of acetic acid, and 100 ml of water was initially charged. 529 g of a 40% by weight aqueous solution of methylglyoxal (2.94 mol) was added with cooling (8-12° C.). The mixture was then heated under reflux for four hours. The mixture was cooled and extracted with four 1.5 I portions of ethyl acetate. The combined organic extracts were dried over sodium sulfate. The organic solvent was then removed under reduced pressure. The crude product was purified by column chromatography (silica; cyclohexane/ethyl acetate). 21 g (approx. 100% by weight, 0.133 mol) of a pale yellow solid was obtained (3-acetyl-4-hydroxy-1,5-dimethylpyrazole).

Variant B: A solution of 1.47 g (10 mmol) of methylhydrazine sulfate and 10 ml of water was initially charged. After heating to 50° C., 3.04 g (25 mmol) of methylglyoxal 1,1-dimethylacetal was added and the mixture was then stirred at 58ºC for 21 hours. The mixture was cooled and adjusted to pH 7.0 with aqueous sodium hydroxide solution (25% by weight). The mixture was extracted with six 8 ml portions of ethyl acetate. The combined organic extracts were dried over magnesium sulfate. The organic solvent was then removed under reduced pressure. 1.44 g (approx. 78% by weight, 7.3 mmol) of a pale yellow solid was obtained (3-acetyl-4-hydroxy-1,5-dimethylpyrazole).

Variant C: A solution of 11 g (75 mmol) of methylhydrazine sulfate and 80 ml of water was initially charged. After heating to 40° C., 34 g of 40% by weight of aqueous methylglyoxal (188 mmol) was added and the mixture was then stirred at 55° C. for 21 hours. The mixture was cooled and adjusted to pH 7.0 with aqueous sodium hydroxide solution (25% by weight). The mixture was extracted with two 200 ml portions of n-butyl acetate. The combined organic extracts were dried over magnesium sulfate. The organic solvent was then removed under reduced pressure. 8 g (approx. 70% by weight, 36 mmol) of a pale yellow solid was obtained (3-acetyl-4-hydroxy-1,5-dimethylpyrazole).

An autoclave was charged with 7.5 g (48.9 mmol) of the pale yellow solid, 2.0 g of Raney nickel, and 60 ml of dioxane and pressurized with 66 bar of hydrogen. The mixture was then heated to 170° C. for three hours with stirring. After cooling and releasing the pressure, the mixture was fil-tered and the organic solvent removed under reduced pressure. The crude product was purified by column chromatography (silica; cyclohexane/ethyl acetate). 4.0 g (28.5 mmol) of a pale yellow solid was obtained (3-acetyl-4-hydroxy-1,5-dimethylpyrazole).

Preparation of 1-tert-butyl-3-ethyl-4-hydroxy-5-methylpyrazole

A solution of 24 g (0.189 mmol) of tert-butylhydrazine hydrochloride and 100 ml of water was initially charged. 136 g of a 40% by weight aqueous solution of methylglyoxal (0.754 mol) and 17 g (0.283 mol) of acetic acid were added. The mixture was then heated under reflux for six hours. The mixture was cooled and extracted with three 0.5 I portions of ethyl acetate. The combined organic extracts were washed with 0.5 I of saturated sodium chloride solution and dried over sodium sulfate. The organic solvent was then removed under reduced pressure. The crude product was purified by column chromatography (silica; cyclohexane/ethyl acetate). 20.6 g (0.106 mol) of a pale yellow solid was obtained (3-acetyl-1-tert-butyl-4-hydroxy-1-methylpyrazole).

An autoclave was charged with 2.5 g (12.8 mmol) of the pale yellow solid, 0.8 g of Raney nickel, and 60 ml of dioxane and pressurized with 61 bar of hydrogen. The mixture was then heated to 170° C. for three hours with stirring. After cooling and releasing the pressure, the mixture was fil-tered and the organic solvent removed under reduced pressure. The crude product was purified by column chromatography (silica; cyclohexane/ethyl acetate). 1.5 g (8.2 mmol) of a pale yellow solid was obtained (1-tert-butyl-3-ethyl-4-hydroxy-5-methylpyrazole).

### Inhibition of Polymerization Experiments

The employed monomer was distilled twice to remove the polymerization inhibitor MEHQ. The stated amount of the specified polymerization inhibitor was then in each case added to the monomer obtained.

0.5 ml of each mixture was transferred to a 1.8 ml ampoule and stored at the reported temperature in an air-circulation oven.

In each test series at least three ampoules of each mixture were filled and tested, with the aver-age time for complete polymerization determined by visual examination.

The relative efficacy is calculated as the ratio of the time until polymerization of the sample comprising the polymerization inhibitor being tested and the time until polymerization of the sample comprising MEHQ. The efficacy of MEHQ itself is thus 100%.

The results are compiled in Tables 1 and 2.

TABLE 1
Mixtures comprising acrylic acid

| Polymerization | | | Time until | |
|---|---|---|---|---|
| inhibitor | Amount | Temperature | polymerization | Efficacy |
| MEHQ*) | 0.004 wt % | 80° C. | 14.0 h | 100% |
| EHDMP | 0.004 wt % | 80° C. | 17.9 h | 128% |
| BEHMP | 0.004 wt % | 80° C. | 45.0 h | 321% |
| HDMPP*) | 0.004 wt % | 80° C. | 10.2 h | 73% |
| HMDPP*) | 0.004 wt % | 80° C. | 11.1 h | 79% |

TABLE 2
Mixtures comprising methacrylic acid

| Polymerization | | | Time until | |
|---|---|---|---|---|
| inhibitor | Amount | Temperature | polymerization | Efficacy |
| MEHQ*) | 0.003 wt % | 80° C. | 39.2 h | 100% |
| EHDMP | 0.003 wt % | 80° C. | 44.0 h | 112% |
| BEHMP | 0.003 wt % | 80° C. | 42.9 h | 109% |

MEHQ: Hydroquinone monomethyl ether
EHDMP: 3-Ethyl-4-hydroxy-1,5-dimethylpyrazole
BEHMP: 1-tert-Butyl-3-ethyl-4-hydroxy-5-methylpyrazole
HDMPP: 4-Hydroxy-3,5-dimethyl-1-phenylpyrazole
HMDPP: 4-Hydroxy-3-methyl-1,5-diphenylpyrazole
*)comparative

The polymerization inhibitors of the invention exhibit improved efficacy compared to MEHQ.

### Polymerization Experiments

A reaction vessel was initially charged under a nitrogen atmosphere with 450 g of water. The initially charged reaction mixture was heated to 95° C. with stirring. On reaching a temperature of 95° C., three streams were metered in with stirring and while maintaining the temperature.

Stream 1: 500 g of acrylic acid, metered in over 5 h

Stream 2: 15 g of sodium hypophosphite in 35 g of deionized water, metered in over 4.75 h

Stream 3: 5 g of sodium peroxodisulfate in 66.4 g of deionized water, metered in over 5.25 h

After addition of the three streams, the reaction mixture was stirred at 95° C. for a further hour. The reaction mixture was then cooled to room temperature and 80 g of water added.

The acrylic acid used in stream 1 was stabilized with 200 ppm by weight of 3-ethyl-4-hydroxy-1,5-dimethylpyrazole or 200 ppm by weight of MEHQ.

The polymers obtained were analyzed by GPC (calibration with Na-PAA standard, eluent 0.01 mol/l phosphate buffer pH 7.4 in dist. water containing 0.01 M NaNa).

TABLE 3
Polymerization of acrylic acid

| Polymerization | | |
|---|---|---|
| inhibitor | Amount | Molar weight |
| MEHQ | 0.020 wt % | 9400 g/mol |
| EHDMP | 0.020 wt % | 9500 g/mol |

MEHQ: Hydroquinone monomethyl ether
EHDMP: 3-Ethyl-4-hydroxy-1,5-dimethylpyrazole

The polymerization inhibitor of the present invention resulted in a polyacrylic acid having a higher molecular weight.

1-15. (canceled)

16. A mixture comprising at least one compound of the general formula (I) 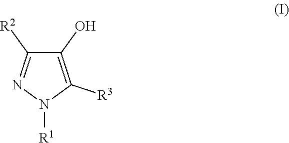
where
R¹ is H or C₁ to C₆ alkyl,
R² is C₂ to C₅ alkyl, and
R³ is C₁ to C₆ alkyl,
and at least one polymerizable compound.

17. The mixture according to claim 16, wherein R¹ is H or C₁ to C₄ alkyl, R² is C₂ to C₄ alkyl, and R³ is C₁ to C₄ alkyl.

18. The mixture according to claim 16, wherein R¹ is H or C₁ to C₄ alkyl, R² is C₂ or C₃ alkyl, and R³ is C₁ to C₃ alkyl.

19. The mixture according to claim 16, wherein R¹ is H or C₁ to C₄ alkyl, R² is C₂ alkyl, and R³ is C₁ or C₂ alkyl.

20. The mixture according to claim 16, wherein the compound of the general formula (I) is 3-ethyl-4-hydroxy-1,5-dimethylpyrazole or 1-tert-butyl-3-ethyl-4-hydroxy-5-methylpyrazole.

21. The mixture according to claim 16, wherein the total amount of compounds of the general formula (I) in the mixture is from 0.0001% to 0.1000% by weight based on the total amount of polymerizable compounds.

22. The mixture according to claim 16, wherein the total amount of the polymerizable compounds is at least 5% by weight based on the total weight of the mixture.

23. The mixture according to claim 16, wherein the polymerizable compound is acrylic acid and/or methacrylic acid.

24. The mixture according to claim 23, wherein the total amount of compounds of the general formula (I) in the mixture is from 0.0050% to 0.1000% by weight based on the total amount of acrylic acid and/or methacrylic acid.

25. The mixture according to claim 16, wherein the polymerizable compound is methyl acrylate, methyl methacrylate, ethyl acrylate, n-butyl acrylate and/or 2-ethylhexyl acrylate.

26. The mixture according to claim 25, wherein the total amount of compounds of the general formula (I) in the mixture is from 0.0001% to 0.0100% by weight based on the total amount of methyl acrylate, methyl methacrylate, ethyl acrylate, n-butyl acrylate and/or 2-ethylhexyl acrylate.

27. The use of at least one compound of the general formula (I) as defined in claim 16 for inhibition of polymerization of at least one polymerizable compound.

28. A process for the storage and/or transport of a mixture according to claim 16.

29. The process according to claim 28, wherein the mixture is stored and/or transported in an oxygen-containing atmosphere.

30. The process according to claim 28, wherein the mixture is stored in a vessel in an oxygen-containing atmosphere having an oxygen content of 5% to 10% by volume and the mixture in the vessel is regularly recirculated.
